(19) United States
(12) Patent Application Publication
(10) Pub. No.: US 2009/0017416 A1
(43) Pub. Date: Jan. 15, 2009

(54) In situ illuminator system for dental use

(76) Inventors: Bao Luu Nguyen, Diamond Bar, CA (US); Liet Hong Nguyen, Garden Grove, CA (US)

Correspondence Address: BLAKELY SOKOLOFF TAYLOR & ZAFMAN LLP, 1279 OAKMEAD PARKWAY, SUNNYVALE, CA, 94085-4040, US

(21) Appl. No.: 12/079,462

(22) Filed: Mar. 26, 2008

Related U.S. Application Data
(60) Provisional application No. 60/920,644, filed on Mar. 28, 2007.

(51) Int. Cl. A61B 1/247; F21V 7/00

(52) U.S. Cl. 433/30; 362/296

(57) ABSTRACT

An embodiment of the invention is an illuminator. A handle has first and second ends. A first light source is attached to the handle the first end. The first light source is powered by an external power source via a wire running through or along the handle.

Another embodiment includes a bite block, a holder embedded in the bite block, and a light source attached to the holder to illuminate an operation field. The light source is powered by a power source.

Another embodiment includes an absorbent pad and an illuminator having a light source attached to the absorbent pad to generate light when powered by a power source.

Another embodiment includes first and second biting pads and a pillar between the first and second biting pads having a length adjusted according to distance between upper and lower teeth.

### RELATED APPLICATIONS

This patent application claims the benefits of U.S. Provisional Application, titled “In situ illuminator system for dental use”, Ser. No. 60/920,644 with a filing date of Mar. 28, 2007.

### BACKGROUND

1. Field of the Invention

Embodiments of the invention relate to the field of dentistry, and more specifically, to dental instrument.

2. Description of Related Art

The classic and most used source of illumination for the dental operating field is the headlight that is part of the dental chair unit. Although it procures a strong light, it cannot reach every area inside the oral cavity, especially toward the back of the mouth. Shadows from the patient's lips and from the operator's head also impede the light to reach the very operating field the dentist is working on.

Existing techniques to solve the problem mentioned in above have a number of drawbacks. One technique uses a small headlight to be mounted directly at the operator's eye glasses, at the level of the nose in between the eyes. That way, the operating field is illuminated without shadow from a light source that is in the vision's axis of the operator. The main drawbacks of such system are: (1) the relatively high cost of the operator's headlight; (2) the fact that the light is in the vision's axis tends to flatten the object's image and impair the operator's sense of depth when working on the tooth, and (3) the system is individually adapted to each operator, and not to the dental unit.

Another technique installs a fiber optic light at the tip of the high-speed hand-piece, illuminating the working area of the bur. The fiber optic is fed with a powerful external light source. The main drawbacks are: (1) the light system is complicated to install, and requires a special kind of hand-pieces (the fiber optic ready handpieces); pushing the cost to a higher level. That installation is dedicated to a specific dental chair unit, and cannot be carried from chair to chair; (2) the fiber optic source is mounted too close to the water spray source of the same handpiece. In some angle of view, the light reflection on the water spray's droplets is more of an inconvenience than a help for the operator.

Yet, another technique uses a cheek or tongue retractor with a fiber optic light system incorporated in the body of those items. The fiber optic is fed with an external powerful light source, similar to the case mentioned above. The main drawback is the cost of a relatively heavy installation. That installation is dedicated to a specific dental chair unit, and cannot be carried from chair to chair.

### BRIEF DESCRIPTION OF THE DRAWINGS

Embodiments of invention may best be understood by referring to the following description and accompanying drawings that are used to illustrate embodiments of the invention. In the drawings:

FIG. 1 is a diagram illustrating a system in which one embodiment of the invention can be practiced.

FIG. 2A is a diagram illustrating a first attachment as a curing unit with one light source according to one embodiment of the invention.

FIG. 2B is a diagram illustrating a first attachment as a curing unit with two light sources according to one embodiment of the invention.

FIG. 2C is a diagram illustrating a first attachment as a curing unit with two light sources and an optical filter according to one embodiment of the invention.

FIG. 3A is a diagram illustrating a second attachment as a hollow enclosure according to one embodiment of the invention.

FIG. 3B is a diagram illustrating a second attachment as an illuminator according to one embodiment of the invention.

FIG. 3C is a diagram illustrating a second attachment as a heating unit according to one embodiment of the invention.

FIG. 3D is a diagram illustrating a second attachment as a vibrator unit according to one embodiment of the invention.

FIG. 3E is a diagram illustrating a second attachment as an illuminator and vibrator unit according to one embodiment of the invention.

FIG. 4A is a diagram illustrating an instrument tip as a spatula according to one embodiment of the invention.

FIG. 4B is a diagram illustrating an instrument tip as a mirror according to one embodiment of the invention.

FIG. 4C is a diagram illustrating an instrument tip as a transluminator according to one embodiment of the invention.

FIG. 4D is a diagram illustrating an instrument tip as a periodontal probe according to one embodiment of the invention.

FIG. 4E is a diagram illustrating an instrument tip as a cement spatula according to one embodiment of the invention.

FIG. 4F is a diagram illustrating an instrument tip as a endodontic explorer according to one embodiment of the invention.

FIG. 4G is a diagram illustrating an instrument tip as a bite bloc according to one embodiment of the invention.

FIG. 4H is a diagram illustrating an instrument tip as a gutta percha heater according to one embodiment of the invention.

FIG. 4I is a diagram illustrating an instrument tip as a wax spatula according to one embodiment of the invention.

FIG. 4J is a diagram illustrating an instrument tip as a heating tip according to one embodiment of the invention.

FIG. 5A is a diagram illustrating an attachment having an illuminating assembly attached to a suction tube according to one embodiment of the invention.

FIG. 5B is a diagram illustrating an attachment having an illuminating assembly attached to a mirror according to one embodiment of the invention.

FIG. 6 is a diagram illustrating an attachment having an imaging device according to one embodiment of the invention.

FIG. 7 is a flowchart illustrating a process to use the multi-functional dental instrument according to one embodiment of the invention.

FIG. 8A is a diagram illustrating a bite block assembly according to one embodiment of the invention.

FIG. 8B is a diagram illustrating a bite block assembly used in a patient's mouth according to one embodiment of the invention.

FIG. 9A is a diagram illustrating an absorbent pad assembly according to one embodiment of the invention.

FIG. 9B is a diagram illustrating an absorbent pad assembly used in a patient's mouth according to one embodiment of the invention.

FIG. 10A is a diagram illustrating an adjustable bite block assembly according to one embodiment of the invention.

FIG. 10B is a diagram illustrating an adjustable bite block assembly used in a patient's mouth according to one embodiment of the invention.

FIG. 11A is a diagram illustrating an illuminator mirror with front lighting, according to one embodiment of the invention.

FIG. 11B is a diagram illustrating an illuminator mirror with back lighting, according to one embodiment of the invention.

FIG. 11C is a diagram illustrating an illuminator mirror with front and back lighting, according to one embodiment of the invention.

FIG. 11D is a diagram illustrating an illuminator mirror with double sided lighting, according to one embodiment of the invention.

FIG. 12A is a diagram illustrating an illuminator stick according to one embodiment of the invention.

FIG. 12B is a diagram illustrating an illuminator stick with a suction tip according to one embodiment of the invention.

FIG. 12C is a diagram illustrating an illuminator stick with two mechanical clips according to one embodiment of the invention.

FIG. 12D is a diagram illustrating an illuminator stick with two LEDs according to one embodiment of the invention.

FIG. 13A is a diagram illustrating a stand-alone illuminator according to one embodiment of the invention.

FIG. 13B is a diagram illustrating the stand-alone illuminator with a flexible arm according to one embodiment of the invention.

### DESCRIPTION

Embodiments of the present invention are illumination units used for dental operating field. One embodiment is an illuminator mirror with front lighting, back lighting, front and back lighting, or double-sided lighting. A handle has first and second ends. A light source is attached to the handle at the first end. The light source is powered by an external power source via a wire running through or along the handle. A mirror is attached to the handle at the first end at an angle. The mirror has a mirror surface facing toward the light source

Another embodiment is an illuminator stick. Another embodiment is a stand-alone illuminator. Another embodiment includes a bite block, a holder embedded in the bite block, and a light source attached to the holder to illuminate an operation field. The light source is powered by a power source. Another embodiment includes an absorbent pad and an illuminator having a light source attached to the absorbent pad to generate light when powered by a power source.

In the following description, numerous specific details are set forth. However, it is understood that embodiments of the invention may be practiced without these specific details. In other instances, well-known circuits, structures, and techniques have not been shown to avoid obscuring the understanding of this description.

One embodiment of the invention may be described as a process which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a program, a procedure, a method of manufacturing or fabrication, etc.

One embodiment of the invention is an in situ powered hand multi-functional dental instrument. The instrument is compact, versatile, and convenient to use. It provides the dentist and his or her dental assistant a versatile tool to perform dental procedures on a patient in a very efficient manner. Another embodiment includes a bite block, a holder embedded in the bite block, and a light source attached to the holder to illuminate an operation field. The light source is powered by a power source. Another embodiment includes an absorbent pad and an illuminator having a light source attached to the absorbent pad to generate light when powered by a power source.

One embodiment of the invention includes an illuminator system that can be easily placed inside the mouth without creating undue inconvenience to the dentist's work, and which illuminates the operating field. The light direction is lateral to the vision's axis, thus enhancing the depth perception when preparing the tooth. The light source is embedded inside the illuminator, and the external power source can be easily carried from one dental chair unit to another. If used for intra-oral photography, this device allows taking pictures without a frontal flash, giving more depth to the image, and solving the eventual shadows from the lips and/or cheeks.

An embodiment of the invention is an illuminator. A handle has first and second ends. A first light source is attached to the handle the first end. The first light source is powered by an external power source via a wire running through or along the handle. Another embodiment includes a bite block, a holder embedded in the bite block, and a light source attached to the holder to illuminate an operation field. The light source is powered by a power source. Another embodiment includes an absorbent pad and an illuminator having a light source attached to the absorbent pad to generate light when powered by a power source. Another embodiment includes first and second biting pads and a pillar between the first and second biting pads having a length adjusted according to distance between upper and lower teeth.

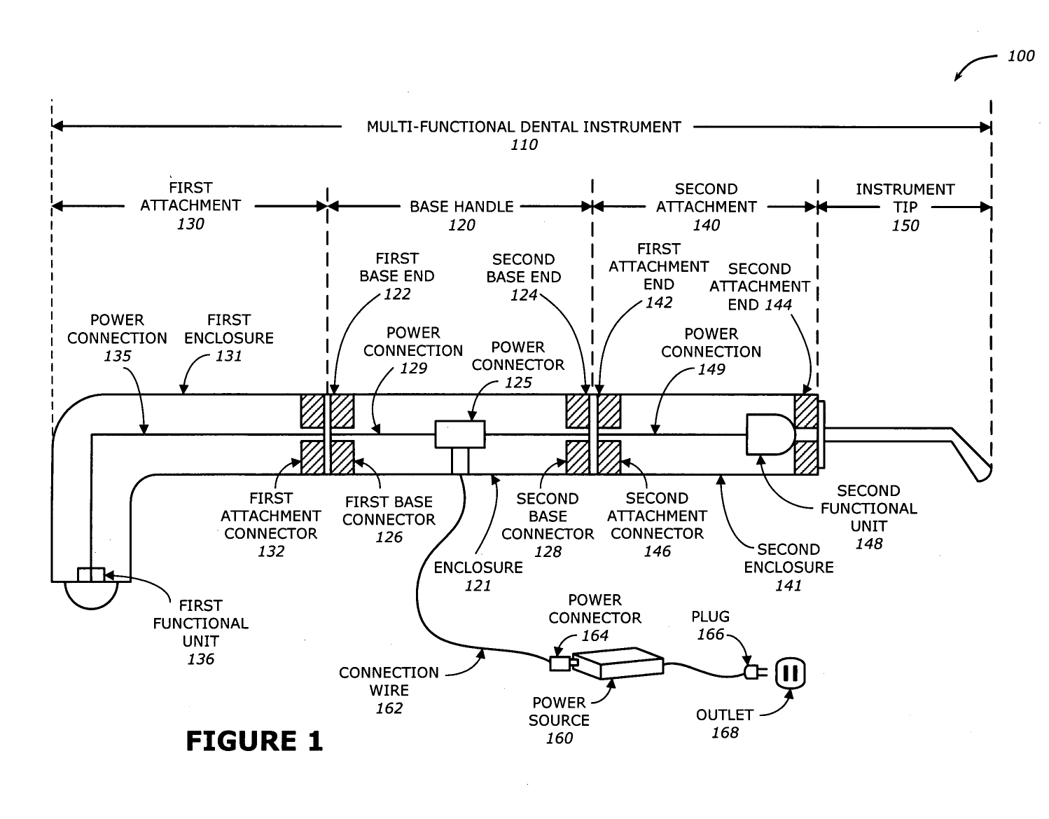

FIG. 1 is a diagram illustrating a system 100 in which one embodiment of the invention can be practiced. The system 100 includes a multi-functional dental instrument 110 and a power source 160. The dental instrument 110 may include a base handle 120, a first attachment 130, a second attachment 140, and an instrument tip 150. Note that the system 100 and/or the dental instrument 110 may include more or less than the above components. For example, the second attachment 140 and the instrument tip 150 may not be needed in some configurations.

The instrument 110 is connected to the power source 160 via a connection wire 162 and a power connector 164. The power source 160 may be any suitable power source that may generate direct current (DC) power to power the instrument 110. The power source 160 may be plugged into an electrical outlet 168 via a plug 166 to convert alternating current (AC) (e.g., 120 VAC to 220 VAC) to a suitable DC voltage (e.g., 9 VDC to 20 VDC). The power source 160 may also be a DC power source (e.g., batteries) located inside the base handle 120 if it is compact enough.

The base handle 120 has a first base end 122 and a second base end 124 opposite to each other. The length of the base handle 120 may be any suitable length. It may range from one inch to six inches. The base handle 120 may include a housing 121 that may be constructed by any suitable material (e.g., plastic, metal, metal alloy, or any combination of them). The housing 121 may be an elongated hollow structure having a cross section of any suitable shape such as circle, hexagonal, square, rectangle, etc. In other words, it may have a shape of a cylinder, a rectangular prism, a hexagonal prism, etc. The diameter of the base handle may be about 7 mm. The housing 121 may house or contain a power connector 125 to connect to the power source 160, a first base connector 126 at the first base end 122, and a second base connector 128 at the second base end 124. The power connector 125 may be located inside the base handle 120. It has power connection 129 to connect the first base connector 126 and the second base connector 128. The power connection 129 may be any suitable conductor to conduct electricity or carry power.

The first attachment 130 is attached to the base handle 120 at the first base end 122. It may be coupled to the first base connector 126 at the first base end 122. The first attachment 130 may be powered by the power source 160 to perform a first dental task at a working area. The first dental task may be any suitable task in a dental procedure (e.g., curing). The working area is typically the area inside the patient's mouth where dental work is being performed.

The first attachment 130 may include a first attachment connector 132, a first enclosure 131, and a first functional unit 136. The first attachment connector 132 may be mechanically compatible with the first base connector 126 such that the first attachment 130 is coupled to the first base connector 126 via a fast action (e.g., a snap action). The first enclosure 131 may be hollow and have a first length. Its shape may be any suitable shape and may be compatible with the base handle 120. For example, its diameter may be approximately 7 mm. The first functional unit 136 may be placed inside the first hollow enclosure 131 and operate by receiving power from the power source 160 via the power connector 125.

The instrument tip 150 is coupled to the base handle 120 directly or indirectly at the first base end 122 (via the first attachment 130) or the second base end 124 (via the second attachment 140) to perform a second dental task different than the first dental task. The instrument tip 150 may be attached directly to the base handle 120 at the second base end 124 via the second base connector 128. It may also be attached indirectly to the base handle 120 through the first attachment 130 or the second attachment 140. By having at least two elements to perform different dental tasks in one instrument, the dental instrument 110 provides convenience and efficiency to the dentist. The dentist may use the first attachment to perform the first task. Then, he or she may turn the instrument around to use the instrument tip 150 to perform the second task.

The second attachment 140 may have a first attachment end 142 and a second attachment end 144 opposite to each other. The first attachment end 142 may be coupled to the second base connector 128 of the base handle 120 at the second base end 124. It may be attached to the instrument tip 150 at the second attachment end 144 to operate the instrument tip 150. Similarly, the first attachment 130 may also be attached to the instrument tip 150 to operate the instrument tip 150.

The second attachment 140 may include a second attachment connector 146, a second enclosure 141, and a second functional unit 148. The second attachment connector 146 may be located at the first attachment end 142 and mechanically compatible with the second base connector 128 of the base handle 120 such that the second attachment connector 146 is coupled to the second base connector 128 via a fast action (e.g., snap action). The second enclosure 141 may be hollow and have a second length which may be the same or different than the first length of the first attachment 130. It may have any suitable shape (e.g., cylinder). The second functional unit 148 may be placed inside the second enclosure 141 and operate by receiving power from the power source 160 via the power connector 125 in the base handle 120.

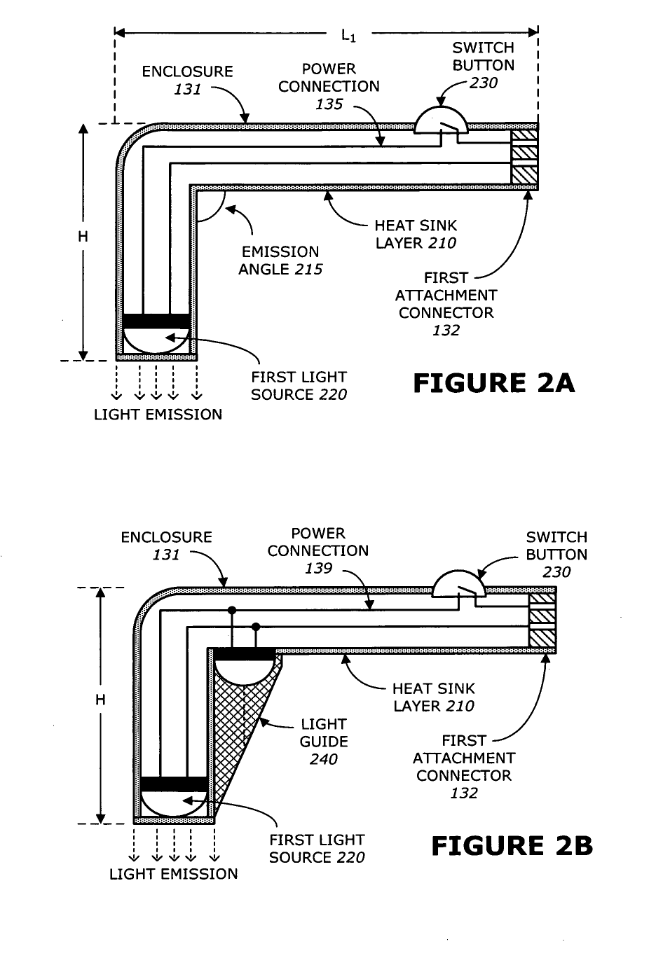

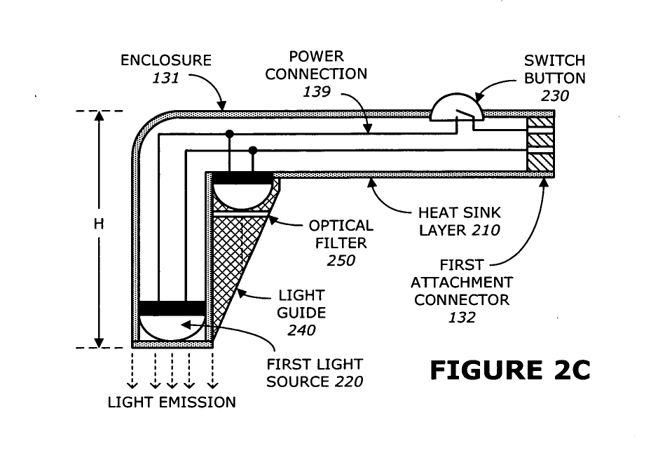

FIG. 2A is a diagram illustrating the first attachment 130 as a curing unit with one light source according to one embodiment of the invention. When the first attachment used as a curing unit together with the instrument tip as a spatula, the combined dental instrument provides a convenient tool for the dentist to perform repetitive intermittent cycles of placement and curing resin composite by just switching the instrument to use the appropriate end.

The first enclosure 131 may be an L-shaped enclosure. It may have a long portion of length L₁ and a short portion of height H. The length L₁ may range from 30 mm to 80 mm. The height H may range from 10 mm to 30 mm. The long portion and the short portion form an emission angle 215. The emission angle 215 may range from 80° to 100°. The enclosure 131 may be made of somewhat flexible material so that the short portion may be bent slightly to adjust the emission angle 215 as necessary to direct the light. The first enclosure 131 may have a heat sink layer 210 that is extended along both sides of the long and short portions. The heat sink layer 210 may include a layer of non thermally conducting material such as epoxy.

The first functional unit 136 may be curing unit having a first light source 220 emitting at a wavelength suitable for curing. The first light source 220 may be a blue LED at a wavelength from 400 nm to 480 nm. The blue LED may have a high power of over 800 mW/cm². The high power of the blue LED facilitates the curing process. The first enclosure 131 may have a heat sink layer 210 to dissipate the heat generated by the light source 220.

FIG. 2B is a diagram illustrating the first attachment 130 as a curing unit with two light sources according to one embodiment of the invention.

In this embodiment, the first functional unit 136 may be the same as in FIG. 2A except that there is a second light source 230 and a light guide 240 located at the corner where the long portion and the short portion meet. The second light source 230 may be a violet LED at a wavelength from 380 nm to 420 nm. The light guide 240 directs the second light source 230 to emit light toward the tip of the short portion so that the two light emissions of the first and second light sources 220 and 230 may be combined, mixed, or fused together.

FIG. 2C is a diagram illustrating the first attachment 130 as a curing unit with two light sources and an optical filter according to one embodiment of the invention.

In this embodiment, the first functional unit 136 may be the same as in FIG. 2B except that the second light source 230 may be a white LED and there is a violet optical filter 250 placed in front of the second light source. The effect is to create a violet light similar to the embodiment shown in FIG. 2B.

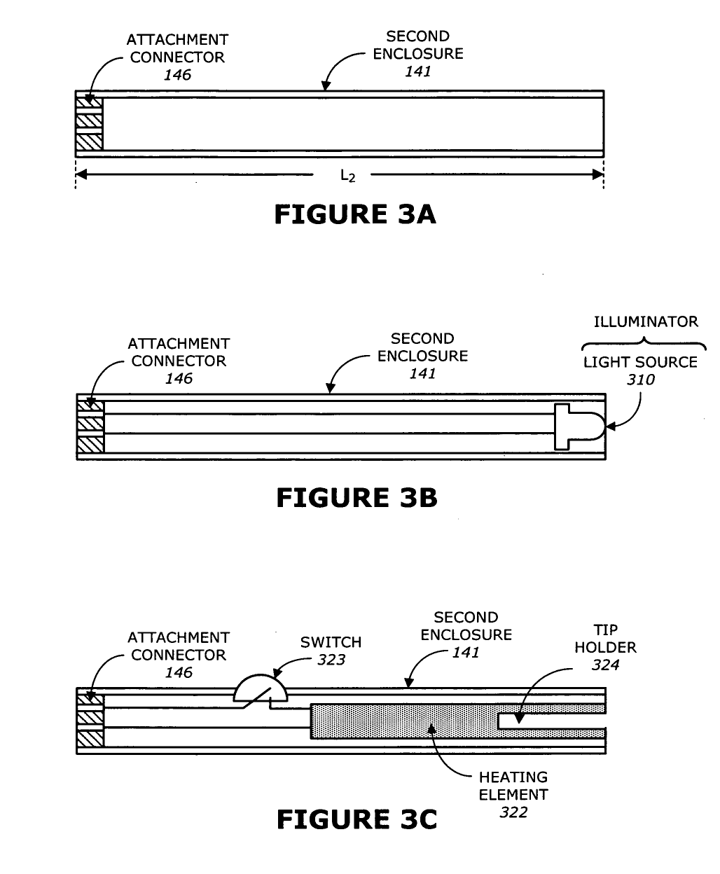

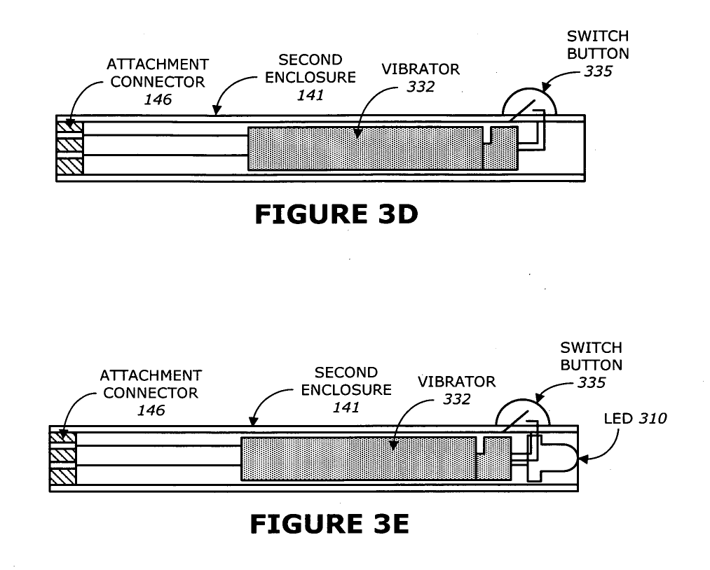

FIG. 3A is a diagram illustrating the second attachment 140 as a hollow enclosure according to one embodiment of the invention.

The second enclosure 141 may be hollow having a length L₂. The length L₂ may range from 30 mm to 80 mm. The second attachment 140 in this embodiment may serve as an extension to the base handle 120.

FIG. 3B is a diagram illustrating the second attachment 140 as an illuminator according to one embodiment of the invention.

In this embodiment, the second functional unit 148 may be an illuminator to emit light to the working area to provide visual comfort to the dentist or the operator using the instrument. It may include a light source 310 extending to the second attachment end 144. The light source 310 may be an LED with sufficient brightness.

FIG. 3C is a diagram illustrating the second attachment 140 as a heating unit according to one embodiment of the invention.

In this embodiment, the second functional unit 148 may be a heating unit to generate heat. It may include a heating element 322 and a switch 323. The heating element 322 may be a heating generating element such as a resistor with low resistance. It may have a tip holder 324 to hold an instrument tip so that the heat may be transferred to the instrument tip. The switch 323 is used to connect or disconnect the electric conduction to the heating element 322 from the power source 160. The switch 323 may be conveniently located on the second enclosure 141 for easy accessibility. The heating unit provides heat to the instrument tip 150 when it is held by the tip holder. This method of heating avoids the inconvenience and potential hazard of using an alcohol lamp or a gas torch to heat the instrument.

FIG. 3D is a diagram illustrating the second attachment 140 as a vibrator unit according to one embodiment of the invention.

In this embodiment, the second functional unit 148 may be a vibrator unit to generate a vibrating or shaking action. It may include a vibrator 332 and a switch 335. The vibrator 332 vibrates when activated. The vibrator 332 may have a motor to control the vibrating movement. The switch 335 is used to connect or disconnect the electric conduction to the vibrator 332 from the power source 160. It may be conveniently located on the second enclosure 141 for easy accessibility. When an instrument tip is attached to the vibrator, the vibrating action caused by the vibrator helps making the material in contact with the instrument tip more flowable, such as composite flowing more easily in deep and narrow spaces, or cement to flow thinly and uniformly when cementing a crown.

FIG. 3E is a diagram illustrating the second attachment 140 as an illuminator and vibrator unit according to one embodiment of the invention.

In this embodiment, the second functional unit 148 includes both the functionalities of an illuminator and a vibrator. It may include the illuminator 310 as in FIG. 3B, the vibrator 332 as in FIG. 3D, and a switch 335. The switch 335 is used to connect or disconnect the electric conduction to the illuminator 310 and the vibrator 332 from the power source 160. It may be conveniently located on the second enclosure 141 for easy accessibility.

Any combination of the above embodiments may be realized. For example, the heating element in FIG. 3C may be combined with the illuminator (FIG. 3B) or the vibrator (FIG. 3D).

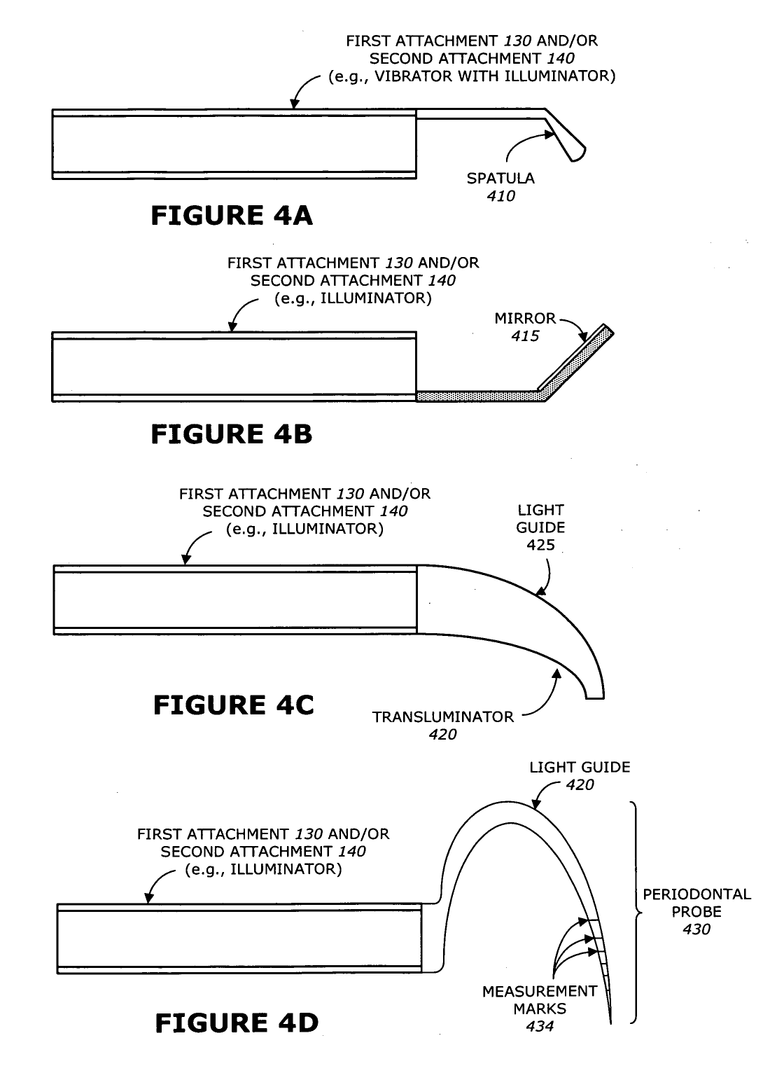

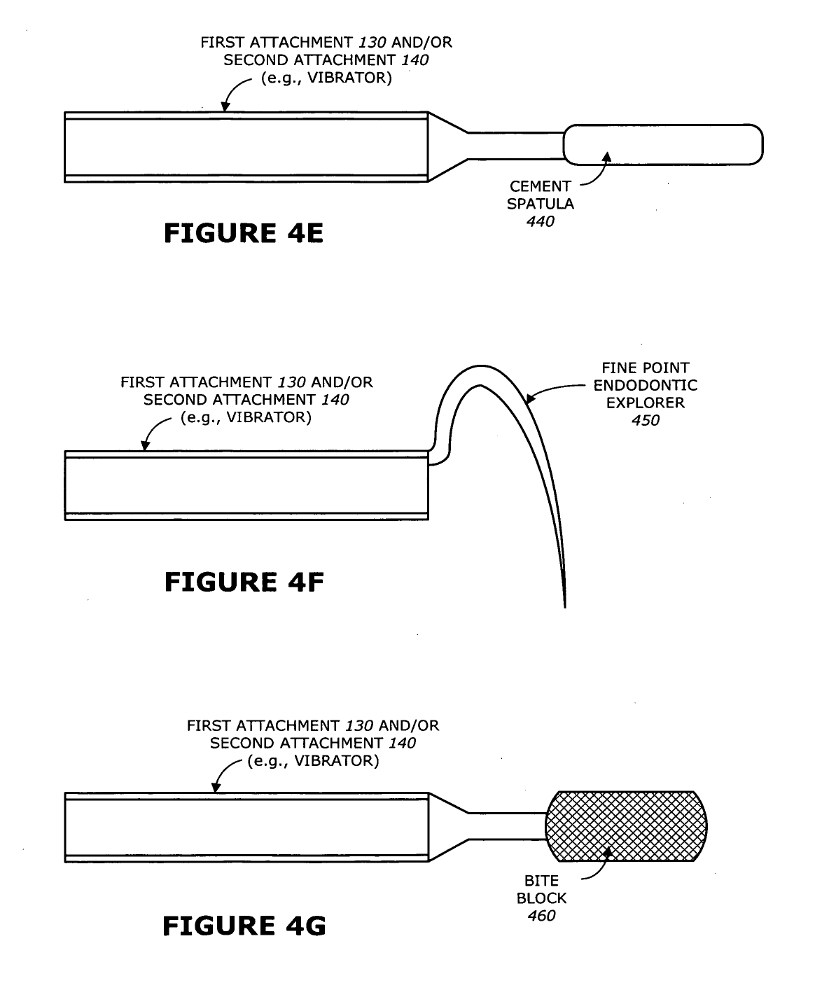

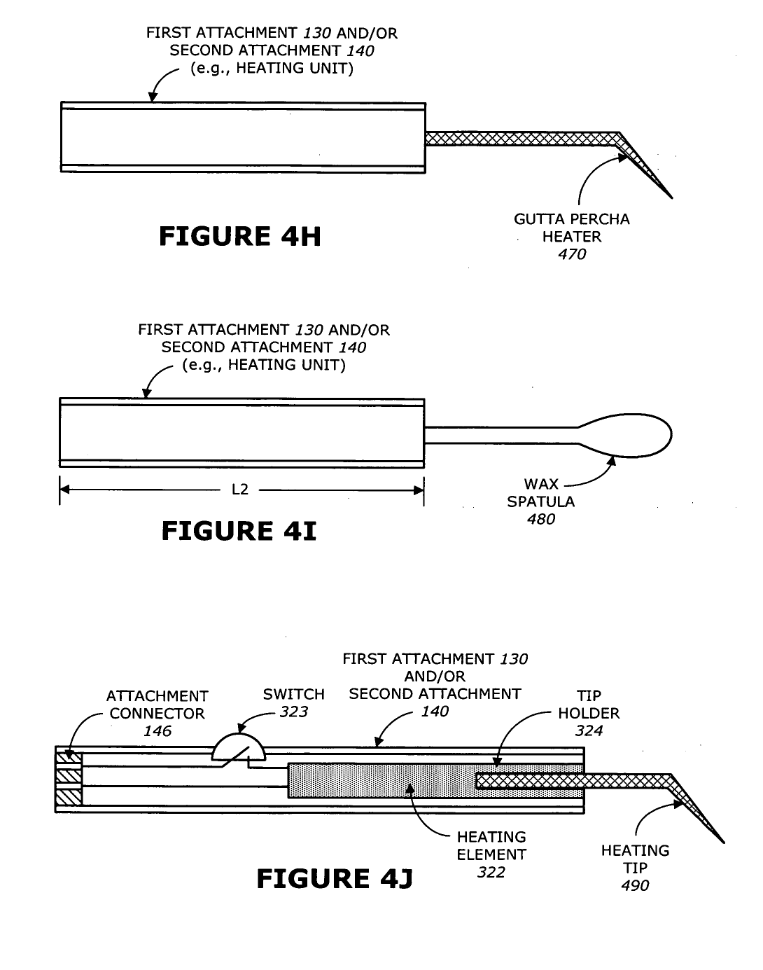

FIG. 4A is a diagram illustrating the instrument tip 150 as a spatula according to one embodiment of the invention. The instrument tip 150 may be attached to the second attachment 140. In this embodiment, the instrument tip 150 may be a spatula 410 attached to the second attachment end 144 of the second attachment 140, or the first attachment 130. The second attachment 140 may be the vibrator with illuminator. The housing of the second attachment 140 may be made of a hard material to maximize the transmission of the vibrations from the vibrator. The spatula 140 is used to place a filling material. It may be attached to the second attachment 140 at the edge of the enclosure.

FIG. 4B is a diagram illustrating the instrument tip 150 as a mirror according to one embodiment of the invention. The instrument tip 150 may be attached to the second attachment 140. In this embodiment, the instrument tip 150 may be a mirror 415 attached to the second attachment end 144 of the second attachment 140. The second attachment 140 may be the illuminator as shown in FIG. 3B. The enclosure of the second attachment 140 may be transparent and have a closed end toward the instrument tip.

FIG. 4C is a diagram illustrating the instrument tip 150 as a transluminator according to one embodiment of the invention. In this embodiment, the instrument tip 150 may be a transluminator 420 attached to the second attachment end 144 of the second attachment 140. The second attachment 140 may be the illuminator as shown in FIG. 3B. The transluminator 420 may have a light guide to direct the light from the second attachment 140 to the tip of the transluminator 420.

FIG. 4D is a diagram illustrating the instrument tip 150 as a periodontal probe according to one embodiment of the invention. In this embodiment, the instrument tip 150 may be a periodontal probe 430 attached to the second attachment end 144 of the second attachment 140. The second attachment 140 may be the illuminator as shown in FIG. 3B. The periodontal probe 430 may include a light guide 432 and measurement marks 434. The light delivered by the illuminator provides visual comfort for the dentist or the operator when measuring the periodontal pockets around the gum of the patient.

FIG. 4E is a diagram illustrating the instrument tip 150 as a cement spatula according to one embodiment of the invention. The instrument tip 150 may be attached to the second attachment 140. In this embodiment, the instrument tip 150 may be a cement spatula 440 attached to the second attachment end 144 of the second attachment 140. The second attachment 140 may be the vibrator as shown in FIG. 3D. The vibration caused by the vibrator allows a fast mixing of cement powder and liquid, leading to a homogeneous result.

FIG. 4F is a diagram illustrating the instrument tip 150 as an endodontic explorer according to one embodiment of the invention. The instrument tip 150 may be attached to the second attachment 140. In this embodiment, the instrument tip 150 may be a fine point endodontic explorer 450 attached to the second attachment end 144 of the second attachment 140. The second attachment 140 may be the vibrator as shown in FIG. 3D. When placing cement or sealer material inside the root canal with the explorer, the vibration caused by the vibrator makes the material more flowable and allows a more complete spread inside the canal than without the vibrator. It may be attached to the second attachment 140 at the edge of the enclosure.

FIG. 4G is a diagram illustrating the instrument tip 150 as a bite bloc according to one embodiment of the invention. The instrument tip 150 may be attached to the second attachment 140. In this embodiment, the instrument tip 150 may be a bite block 460 attached to the second attachment end 144 of the second attachment 140. The bite block 460 may be made of soft plastic. The second attachment 140 may be the vibrator as shown in FIG. 3D. The enclosure of the second attachment 140 may be made of a hard material. When cement a crown, the patient is asked to bite on an object (e.g., a cotton roll, a soft plastic bite block) to exert a pressure on the crown while the cement has not become hard yet. The vibrator may vibrate for a few seconds at the beginning of the pressure. This vibrating action causes the cement to flow more thinly under the crown and allows a better fit at the crown's margins.

FIG. 4H is a diagram illustrating the instrument tip 150 as a gutta percha heater according to one embodiment of the invention. The instrument tip 150 may be attached to the second attachment 140. In this embodiment, the instrument tip 150 may be a gutta percha heater 470 attached to the second attachment end 144 of the second attachment 140. The second attachment may be the heater unit shown in FIG. 3C. The heating element in the heater unit delivers the necessary hear to cut the gutta percha which is the root canal main filling core material.

FIG. 4I is a diagram illustrating the instrument tip 150 as a wax spatula according to one embodiment of the invention. The instrument tip 150 may be attached to the second attachment 140. In this embodiment, the instrument tip 150 may be a wax spatula 480 attached to the second attachment end 144 of the second attachment 140. The second attachment may be the heater unit shown in FIG. 3C. The heating element in the heater unit delivers the necessary hear to work on wax for denture try-in sessions.

FIG. 4J is a diagram illustrating the instrument tip 150 as a heating tip according to one embodiment of the invention. In this embodiment, the instrument tip 150 may be a heating tip 490 attached to the second attachment end 144 of the second attachment 140. The second attachment 140 may include the heating unit as shown in FIG. 3C. The heating tip 490 may be the gutta percha heater or the wax spatula shown in FIGS. 4H and 4I. It may be held at the tip holder 324 of the heating element 322.

A number of configurations to enhance the versatility of the first attachment 130 may be made. The first attachment 130 may include an illuminating unit and a supplementary device having a tip attached to the first enclosure or the illuminating assembly such that the illuminating assembly is positioned at an illuminating distance from the tip.

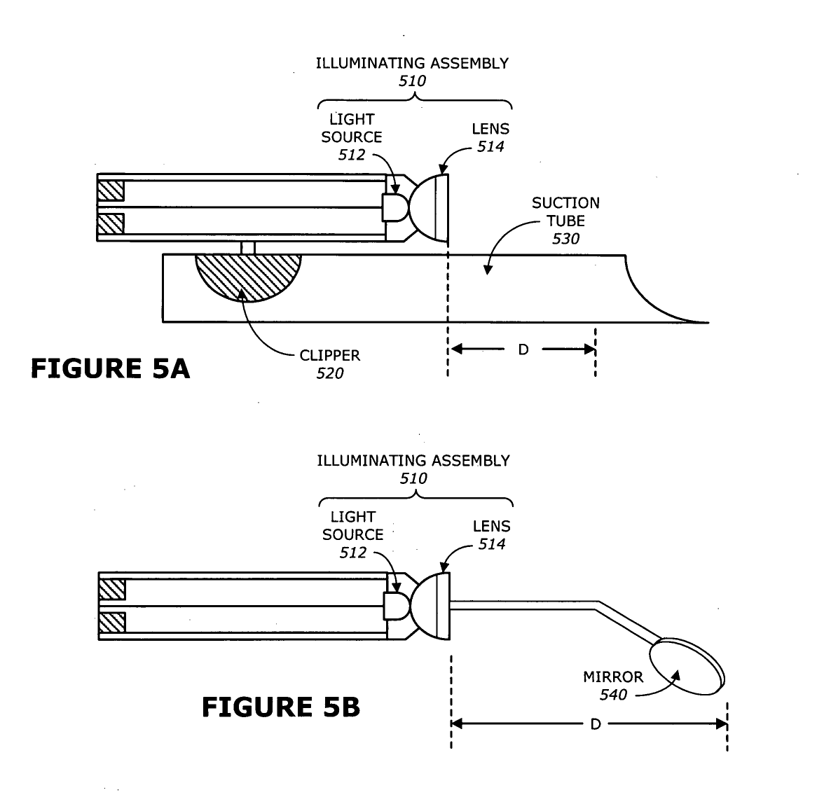

FIG. 5A is a diagram illustrating the first attachment 130 having an illuminating assembly attached to a suction tube according to one embodiment of the invention. The functional unit 136 of the first attachment 130 may be an illuminating assembly 510. The illuminating assembly 510 may include a light source 512 and a lens 514. The light source 512 may be an LED inserted into or attached to the lens 514. The lens 514 may be selected to have a suitable focal length to focus the light to the object being illuminated in the working area. The light intensity therefore is increased. The supplementary device may be a suction tube 530. The suction tube 530 may be firmly attached to the first attachment 130 by a clipper 520. The illuminating distance D between the tip of the suction tube 530 and the illuminating assembly 510 may range from three to eight inches.

FIG. 5B is a diagram illustrating an attachment having an illuminating assembly attached to a mirror according to one embodiment of the invention.

In this embodiment, the illuminating assembly is the similar to the illuminating assembly 510 in FIG. 5A. A mirror 510 may be attached to the center of the illuminating assembly 510. The focused light from the illuminating assembly 510 illuminates directly the area behind the back of the mirror 510 and also reflects the light on the mirror surface. In this manner, the instrument allows illumination in both direct and indirect examinations.

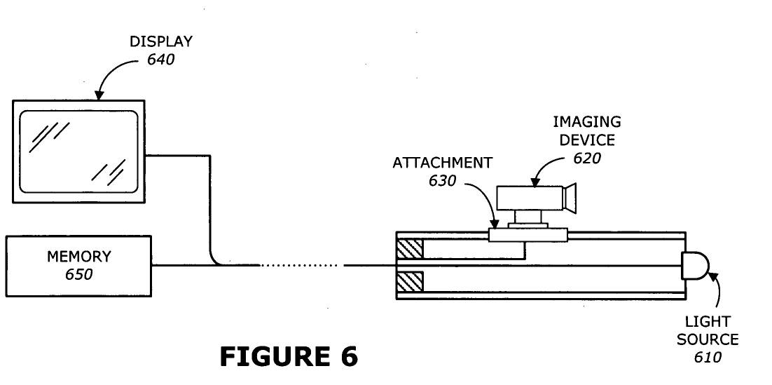

FIG. 6 is a diagram illustrating an attachment having an imaging device according to one embodiment of the invention.

The attachment, either the first attachment 130 or the second attachment 140, has a light source 610 and an imaging device 620. The light source 610 may be an LED with sufficient brightness to provide light for the imaging process. The imaging device 620 is attached to the first enclosure 131 by an attachment mechanism 630 to capture images at the work area. The imaging device 620 acts as an intra-oral camera to capture intra-oral images. The captured images may then be transmitted to a display screen 640 located externally to the first attachment 130. The captured images may also be stored in a memory 650. The imaging device 620 may be a miniature camera or video camera. The display screen may be a small (e.g., 2-inch, 3-inch, or 5-inch) liquid crystal display (LCD) or any other suitable display device. The memory 650 may include a memory card reader or a flash disk device with universal serial bus (USB) interface, such as a high capacity (e.g., 4 GB-16 GB) memory stick. The display screen 640 and the memory 650 may be part of a computer system with processor, memory, input/output devices, etc. Other components such as a frame capture device or imaging processing and analysis software may be employed to analyze or enhance the captured images. The memory 650 may then be transported to transfer to other devices or systems. The display screen 640 may also be used to display the real-time captured images or the stored images. The stored images may be any images including X-ray images and the captured images. The display screen 640 may also be used as a back lit screen for dental X-ray images.

Note that the designation of the first attachment 130 and the second attachment 140 us merely for clarity and convenience. Any one of the first and second attachments 130 and 140 may have the functional unit as described in FIGS. 2A, 2B, 2C, 3A through 3E. Furthermore, the instrument tip 150 may be attached to either the first attachment 130, or the second attachment 140, or both (using two different, or the same, instrument tips). For example, the first attachment 130 may be an illuminator attached to a mirror and the second attachment 140 may be a heater attached to a gutta percha heater.

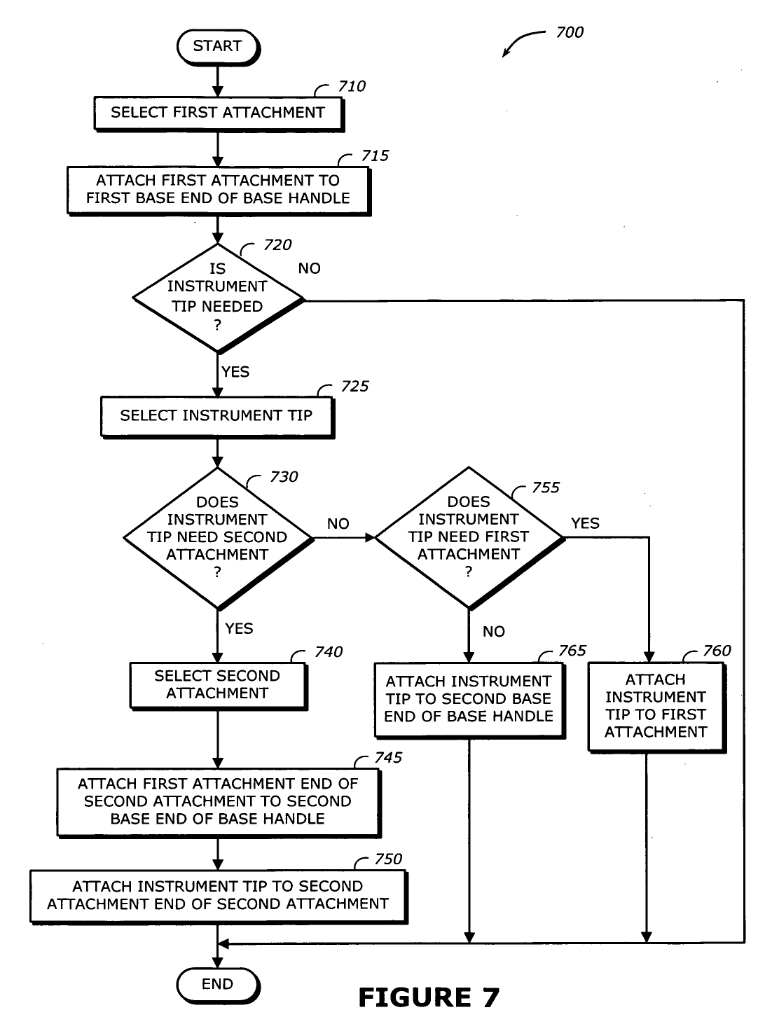

FIG. 7 is a flowchart illustrating a process 700 to use the multi-functional dental instrument according to one embodiment of the invention.

Upon START, the process 700 selects a first attachment from a group of attachments depending on the need (Block 710). For example, the first attachment may be a curing unit, or with a functional unit as an illuminator, a heating unit, a vibrator unit, or a vibrator and illuminator unit, etc.

Then, the process 700 attaches the first attachment to a first base end of a base handle (Block 715). The attachment may be performed using the snap-on connectors at the ends of the first attachment and the base handle.

Next, the process 700 determines if the instrument tip is needed (Block 720). If not, the process 700 is terminated. Otherwise, the process 700 selects the instrument tip to be used with the dental instrument (Block 725). The instrument tip may be selected from tips such as a spatula, a mirror, a transluminator, a periodontal probe, a cement spatula, an endodontic explorer, a crown cementation bite bloc, a gutta percha heater, a wax spatula, and a heating tip.

Then, the process 700 determines if the instrument tip needs a second attachment (Block 730). For example, a periodontal probe may need an illuminator. If not, the process 700 determines if the instrument tip needs the first attachment tip (Block 755). If so, the process 700 attaches the instrument tip to the first attachment (Block 760) and is then terminated. Otherwise, the process 700 attaches the instrument tip directly to a second base end of the base handle (Block 735) and is then terminated. If the instrument tip needs the second attachment, the process 700 selects a second attachment suitable for the instrument tip (Block 740). The process 700 may select a second attachment with a functional unit as an illuminator, a heating unit, a vibrator unit, or a vibrator and illuminator unit, etc.

Then, the process 700 attaches a first attachment end of the selected second attachment to a second base end of the base handle (Block 745). The attachment may be performed using the snap-on connectors at the ends of the second attachment and the base handle. Next, the process 700 attaches the selected instrument tip to a second attachment end of the selected second attachment (Block 750). The process 700 is then terminated.

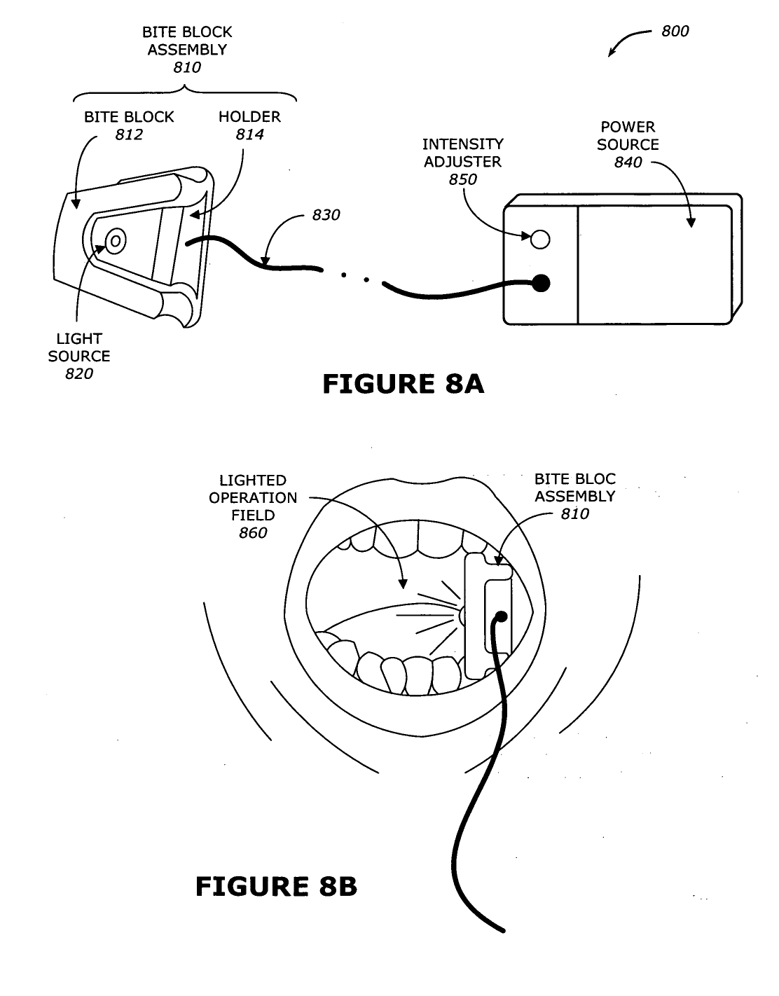

FIG. 8A is a diagram illustrating a bite block unit 800 according to one embodiment of the invention. The bite block unit 800 includes a bite block assembly 810, a light source 820, and a power source 840.

The bite block assembly 810 may be put between the patient's jaws while the patient bites on it. The bite block assembly 810 may be typically put on the opposite side of the operating field. For example, if the dentist works on the patient's left side, the bite block assembly 810 is placed on the patient's right side). The bite block assembly 810 is used to keep the patient's mouth open during the entire operation so that the patient may relax his or her jaw muscles. The bite block assembly 810 includes a bite block 812 and a holder 814. The bite block 812 may be any suitable bite block used in dental work It may be made of relatively soft plastic. The holder 814 provides a mechanical support for the bit block 812. It is embedded in the bite block 812. It may be made of heat-dissipating material and serve as a heat sink for the light source 820. The light source 820 may be a white high intensity LED. It directs the light toward the operating field when the bite block assembly 810 is put in place inside the patient's mouth. The light source 820 may be attached firmly to the holder 814. The holder 814 also provides electrical connections to connect the light source 820 to the power source 840.

The light source 820 obtains power from the power source 840 through a connection wire 830. The power source 840 may be similar to the power source 160 shown in FIG. 1. The power source 840 may have an intensity adjuster 850 to adjust the intensity of the light source 820 by varying the amount of power or current from the power source 830.

FIG. 8B is a diagram illustrating the bite block assembly 810 used in a patient's mouth according to one embodiment of the invention.

The bite block assembly 810 is placed on one side of the patient mouth. The light source 820 directs the light toward the operating field 860 which is on the opposite side of the bite block assembly 810. The bite block assembly 810 therefore serves two purposes: one is to serve as a conventional bite block, and one is to provide illumination inside the patient's mouth.

The bite-bloc assembly 810 may be like the one usually used by dentists to help patients keeping the mouth opened during the dental procedure. The bite bloc assembly 810 may include a super bright light emitting device (LED) mounted on a heat sink; and the whole piece is embedded in a bloc of thermal non conductive material such as epoxy. An electrical wire with connector runs from the bite bloc assembly 810 to outside the mouth, and connects to an external portable power source. The bite-bloc assembly 810 may also be of various designs conceived to minimize the bulkiness, such as a shape covering only one tooth on each jaw, or a V shaped design not incorporating the vertical front part. The LED light bulb, mounted on the heat sink unit, may also be placed at various locations on the bite-bloc assembly 810, such as on the upper part directed from the palate toward the working side, to avoid projecting the shadow of the tongue on the operating field.

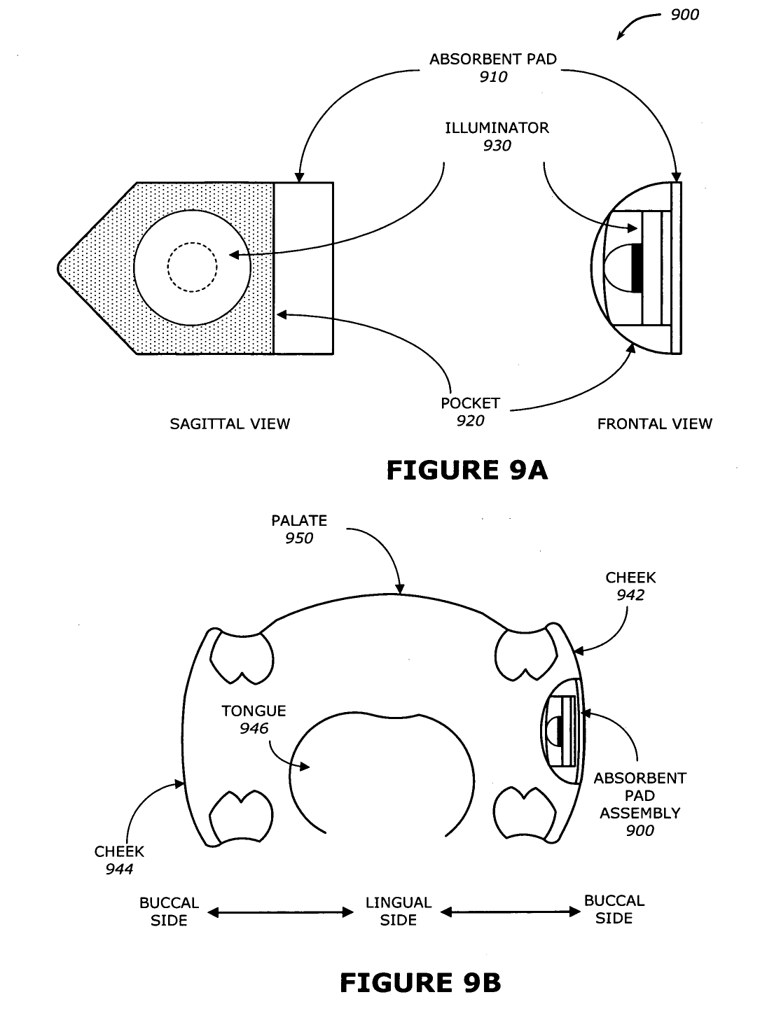

FIG. 9A is a diagram illustrating an illuminator and absorbent pad assembly 900 according to one embodiment of the invention. The illuminator and absorbent pad assembly 900 includes an absorbent pad 910, and illuminator 930, and a pocket 920. The absorbent pad 910 is a saliva absorbent pad. The illuminator 930 may be a light source (e.g., an LED) attached to the absorbent pad to generate light when it is powered by a power source. The power source may be batteries or an external power source. The pocket 920 houses the illuminator 930.

FIG. 9B is a diagram illustrating the illuminator and absorbent pad assembly 900 used in a patient's mouth according to one embodiment of the invention. The absorbent pad assembly 900 described in FIG. 9A may be placed in a patient's mouth to provide light to the working area. The patient's mouth may include cheeks 942 and 944, tongue 946, and palate 950. The illuminator and absorbent pad assembly 900 may be placed in any convenient place. For example, it may be placed on a cheek 942 opposite of cheek 944.

The traditional absorbent pad 910 may include a thin, semi rigid pad in a shape of a triangle or a rectangle with a triangular tip. The pad 910 is placed against the cheek, with the tip directed toward the back. The function of the pad 910 is to absorb the saliva coming from the saliva gland situated on the cheek side, behind the molar. The pad 910 assures a relatively dry environment for the working area. Usually, the absorbent material is covered by a porous plastic film on each side.

The embodiment shown in FIG. 9B may include adding a clear plastic pocket on the saliva absorbent pad 910, on the interior side facing the interior of the mouth when the pad 910 is placed against the cheek. A stand-alone illuminator may be placed inside the above mentioned plastic pocket.

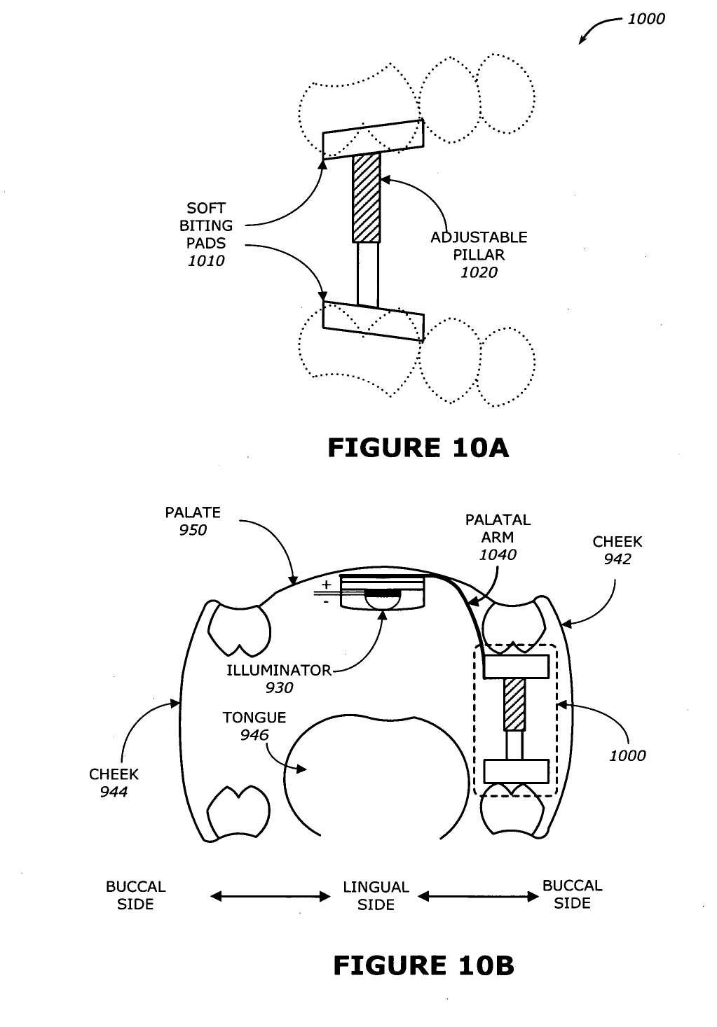

FIG. 10A is a diagram illustrating an adjustable bite block assembly 1000 according to one embodiment of the invention. The adjustable bite block assembly 1000 is shown in a sagittal view. It includes soft biting pads 1010 and adjustable pillar 1020. The adjustable pillar 1020 is positioned between the two soft biting pads 1010. Its length may be adjusted accordingly to create a pressure on the upper and lower teeth when used in a patient's mouth.

The biting pads (upper and lower) 1010 cover only one tooth, instead of three teeth as in a traditional bite-bloc. The advantage is to liberate the area toward the front, in order allow easy access for the operator from that side of the mouth. The biting pads 1010 are connected together by means of a rigid pillar 1020 that is adjustable in height, instead of being part of a unique (rectangular, trapezoidal, or triangular) bloc as in a traditional bite-bloc. The advantage is to have one bite-bloc that can be adjusted to the patient's mouth

FIG. 10B is a diagram illustrating the adjustable bite block assembly 1000 used in a patient's mouth according to one embodiment of the invention. The adjustable bite block assembly 1000 may be used together with the illuminator 930 shown in FIG. 9A. For example, the adjustable bite block assembly 1000 may be placed between the upper and lower teeth on the cheek 942 and the illuminator 930 may be placed on the palate 950. A palate arm 1040 connecting the adjustable bite block assembly 1000 with the illuminator 930. This embodiment uses the adjustable bite-bloc assembly 1000 described above whereas a flexible palatal arm is located on the lingual part of the upper biting pad, and goes upward toward the palate 950, curving alongside the palate 950; and the stand-alone illuminator 930 is attached to the end of the flexible palatal arm 1040. This embodiment allows the illumination from the roof (the palate) of the mouth, the same way a ceiling light illuminates a room. The illumination from this angle avoids the eventual projection of the shadow of the tongue on the working area.

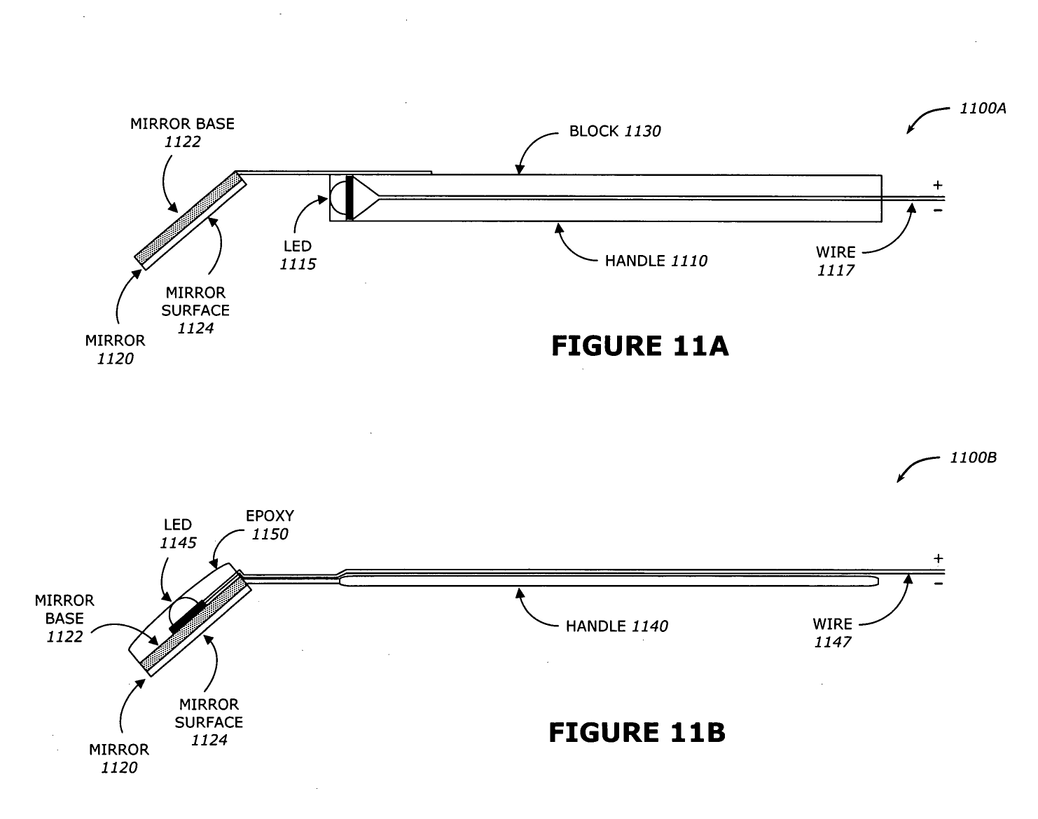

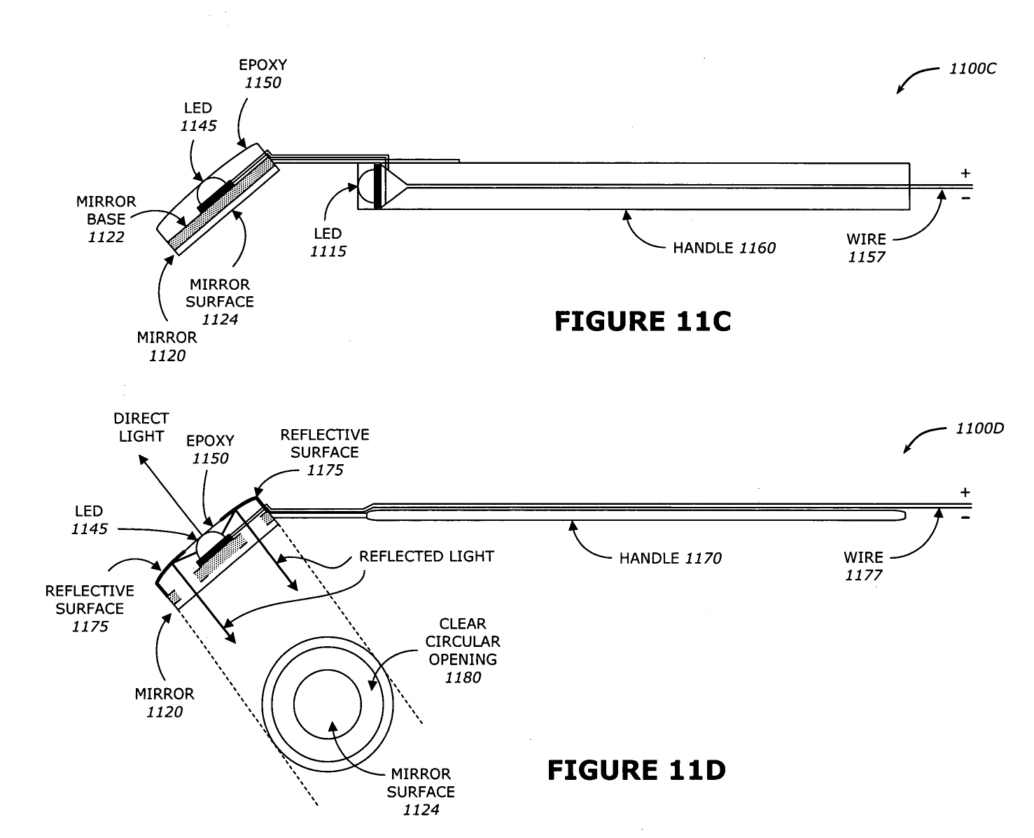

FIG. 11A is a diagram illustrating an illuminator mirror 1100A with front lighting, according to one embodiment of the invention. The illuminator mirror 1100A includes a handle 1110 and a mirror 1120.

The handle 1110 includes a metallic cylinder of about 7 mm in diameter and about 15 cm long, serving also as heat sink for a LED 1115 light bulb that is mounted and the end of the handle 1110, directing the light toward the mirror 1120. It also includes an electrical wire 1117 with connector running from the LED 1115, following (e.g., through or along) the handle 1110, exiting through the end of the handle 1110, and connecting to an external portable power source. A bloc 1130 of thermal non conductive material such as epoxy covering the LED-handle-wire assembly. The dental examination mirror 1120 includes a round regular dental mirror of about 20 mm in diameter, tilted at an angle of about 45 degrees with the axis of the handle 1110, and connected to the handle 1110. It is attached to the handle 1110 via any suitable attachment mechanism. The mirror 1120 includes a mirror base 1122 and a mirror surface 1124. When the LED 1115 is powered, the light reflecting on the mirror surface 1124 illuminates the area to be examined.

FIG. 11B is a diagram illustrating an illuminator mirror 1100B with back lighting, according to one embodiment of the invention. The illuminator mirror 1100B includes a handle 1140 and the dental examination mirror 1120.

The handle 1140 is a handle for the instrument. The mirror 1120 is similar to the mirror 1120 shown in FIG. 11A. It is attached to the handle 1140 via any suitable attachment mechanism. It may be a round regular dental mirror of about 20 mm in diameter, tilted at an angle of about 45 degrees with the axis of the handle 1140, and connected to the handle 1140. A light source (e.g., LED) 1145 is mounted on the metallic base 1122 in the back of the mirror that also serves as heat sink. When powered, the light source 1145 emits light from the back of the mirror 1120. An electrical wire 1147 with connector runs from the light source 1145, following (e.g., through or along) the handle 1140, exiting through the end of the handle 1140, and connecting to an external portable power source. A bloc of thermal non conductive material 1150 such as epoxy covers the light source-metallic assembly back of the mirror 1120.

When the light source 1145 is powered, illumination comes from the back of the mirror 1120. Most of the time, the dental mirror is used by dentists as a cheek or tongue retractor, rather than as a mirror. The reflecting surface of the mirror 1120 is applied against the tissue to be retracted (cheek or tongue), with the back of the mirror 1120 facing the examination or working area. Using as a retractor, this embodiment illuminates directly the operating field.

FIG. 11C is a diagram illustrating an illuminator mirror 1100C with front and back lighting, according to one embodiment of the invention. The illuminator mirror 1100C includes a combination of the above. It includes a mirror 1120 with a light source (e.g., LED) 1145 mounted on the back 1122 and a handle 1160 with a light source (e.g., LED) 1115 mounted at the front end. When the light sources 1115 and 1145 are powered, illumination comes directly from the back of the mirror 1120, and indirectly from the front of the mirror 1120 (the reflective surface).

FIG. 11D is a diagram illustrating an illuminator mirror 1100D with double sided lighting, according to one embodiment of the invention.

The illuminator mirror 1100D includes a variation of the mirror described in FIG. 11A, 11B, or 11C whereas the bloc 1150 of thermal non conductive material such as epoxy covering the light source—back of the mirror 1120 assembly is covered on the circumference, with a 5 mm wide band of reflective layer. The reflective layer directs the light, which is transmitted by the light source laterally through the epoxy, toward the front of the mirror 1120. The front part of the mirror 1120 is made of a reflective surface 1175 in the center, and a clear circular opening or circumference 1180 transparent on the circumference, as wide as the reflective band on the back. This clear circular opening 1180 permits the light reflected on the reflective band from the back to illuminate in the direction of the front.

When the light source 1145 is powered, illumination comes from the back of the mirror 1120, and directed directly toward the back and indirectly toward the front (through the circumferential reflective layer).

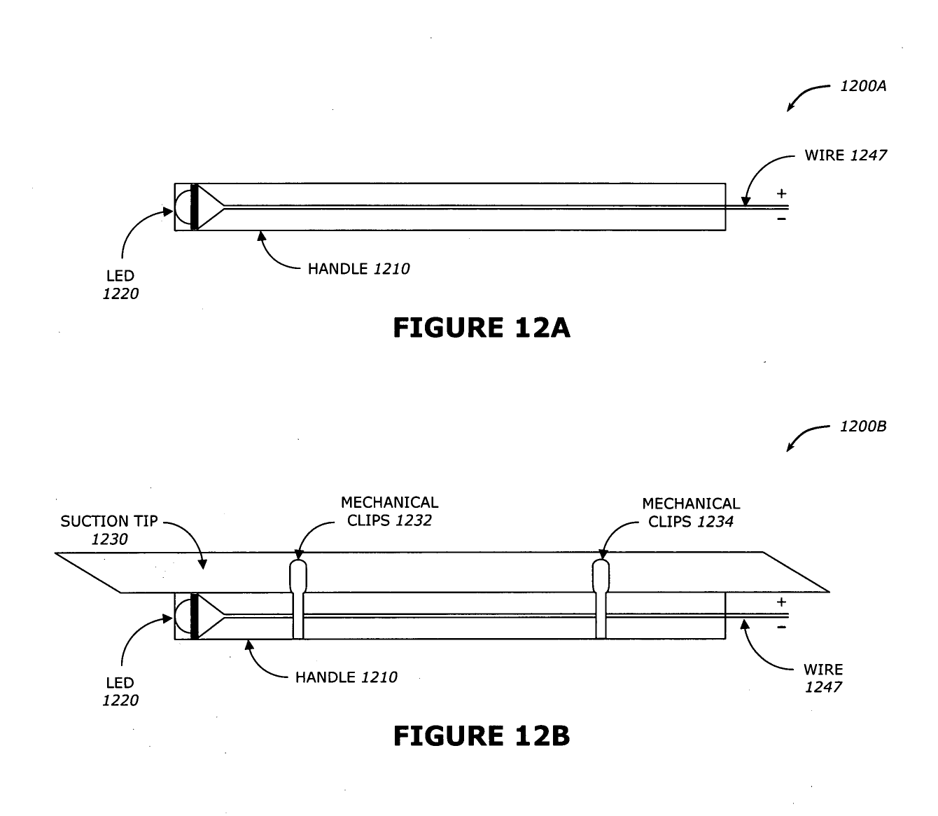

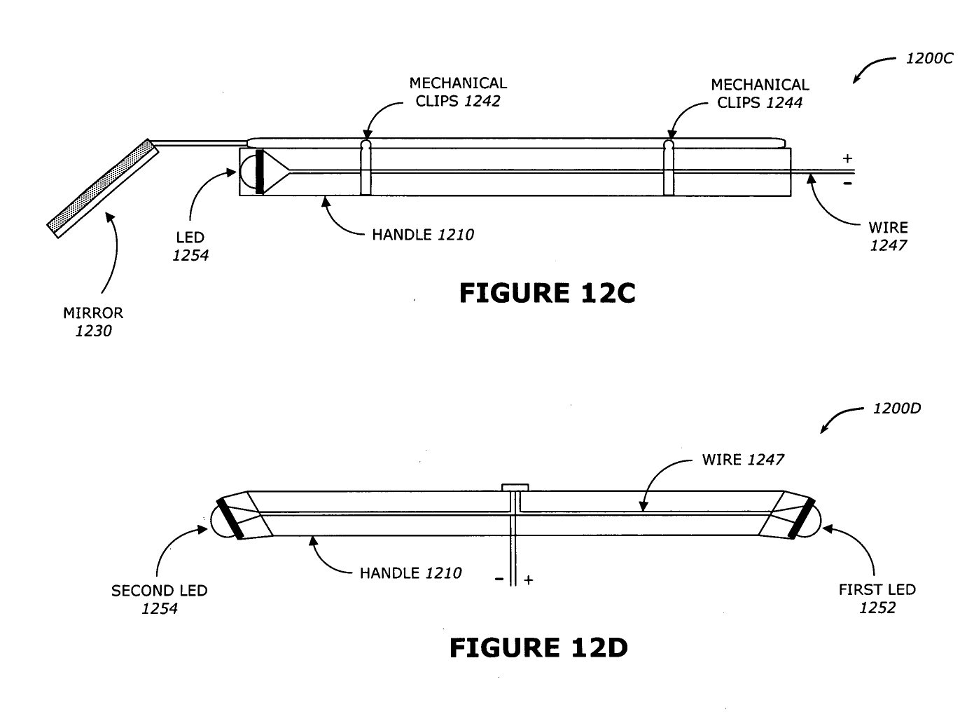

FIG. 12A is a diagram illustrating an illuminator stick 1200A according to one embodiment of the invention. The illuminator stick 1200A includes a handle 1210 and a light source 1220.

The handle 1210 is a stick of metallic cylinder of about 7 mm in diameter and about 12 cm long, serving also as heat sink for the light source (e.g., LED) 1220 that is mounted at one end of the handle 1210. An electrical wire 1227 with connector runs from the light source 1220, following the handle 1210, exiting through the other end of the handle 1210 and connecting to an external portable power source. A bloc of thermal non conductive material such as epoxy covers the light source-handle-wire assembly. The illuminator stick 1200A may be used alone, as a light source to be introduced in the oral cavity to illuminate the operating/examination area.

FIG. 12B is a diagram illustrating an illuminator stick 1200B with a suction tip according to one embodiment of the invention. The illuminator 1200B include the illuminator stick 1200A described in FIG. 12A with an attachment mechanism to attach the handle to a suction tip 1230. The attachment mechanism may include a plurality of clips positioned along the handle 1210. It may include two adapted mechanical clips 1232 and 1234 to attach the handle 1210 to the saliva suction tip 1230. The dental assistant usually assures the saliva suction with the saliva tip right at the operating field. With this embodiment, the operating field is directly illuminated by the illuminator stick attached to the suction tip 1230.

FIG. 12C is a diagram illustrating an illuminator stick 1200C with a mirror and mechanical clips, according to one embodiment of the invention. The illuminator 1200C may be the illuminator stick 1200B as described in FIG. 12B with two adapted mechanical clips 1242 and 1244 to attach the handle 1210 to a dental mirror 1230. This embodiment serves the same purpose as the embodiment 1200A or 1200B, whereas the illuminator stick is used with any regular dental mirror.

FIG. 12D is a diagram illustrating an illuminator stick 1200D with two LEDs designed for translumination exam, according to one embodiment of the invention. The illuminator 1200D may be the illuminator stick 1200A as described in FIG. 12A with a first light source 1252 (e.g., green LED) on one end and a second light source 1254 (e.g., white LED) on the other end. The light sources are mounted at 45 degrees from the axis of the handle 1210. A switch 1260 positioned on the handle 1210 to select one of the first and second light sources for illumination. It allows the selection of the LED to be used. The electrical wire 1257 exits from the middle of the handle 1210. This embodiment is designed for translumination exam.

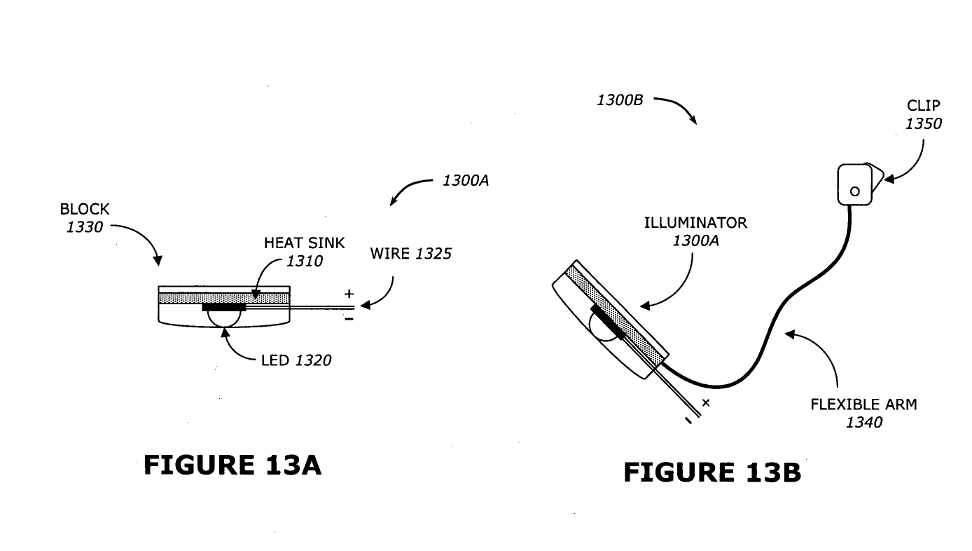

FIG. 13A is a diagram illustrating a main unit of the stand-alone illuminator 1300A according to one embodiment of the invention. The illuminator 1300A includes a heat sink 1310, a LED 1320, and a bloc 1330.

The heat sink 1310 may be a round or oval metallic heat sink of about 15 mm in diameter, 2 mm thick. The LED 1320 is mounted on the heat sink 1310. An electrical wire 1325 with connector runs from the LED 1320, and connecting to an external portable power source. The bloc 1330 of thermal non conductive material such as epoxy covers the LED-metallic heat sink assembly.

The stand-alone illuminator 1300A may be placed in the mouth by means of a denture adhesive to position onto the palate, illuminating the whole oral cavity the way a light bulb on the ceiling illuminates the whole room. The illuminator 1300A may also be placed against the teeth with an adhesive thermoplastic resin bloc as used in dentistry to make the mold for temporary crowns. If the heat sink 1310 is made of a magnetized metal, the illuminator 1300A may be placed on iron steel based material instrument such as a cotton plier, a metallic cheek retractor, etc. It may be introduced in the mouth for illumination. It may also be placed on a metallic dental clamp which is mounted on a tooth, such as in the way the clamp is used to hold the rubber dam in some dental procedures.

FIG. 13B is a diagram illustrating the stand-alone illuminator 1300B attached to a flexible arm according to one embodiment of the invention. The illuminator 1300B may be the stand-alone illuminator 1300A as described in FIG. 13A whereas the illuminator is attached to a flexible arm 1340. The other end of the flexible arm 1340 is stabilized by means of a mechanical clip 1350 to an extra-oral fixed base such as the patient's protective eyeglasses, or the stand-alone saliva suction that is hanging on the patient's lower jaw (contrary to the saliva suction tip that is held by the assistant as described above). The flexible arm 1340 permits the placement of the illuminator 1300A anywhere inside the mouth. The extra-oral fixed base permits the stabilization of the illuminator during the whole period of use.

While the invention has been described in terms of several embodiments, those of ordinary skill in the art will recognize that the invention is not limited to the embodiments described, but can be practiced with modification and alteration within the spirit and scope of the appended claims. The description is thus to be regarded as illustrative instead of limiting.

What is claimed is:

1. An apparatus comprising:
a handle having first and second ends; and
a first light source attached to the handle the first end, the first light source being powered by an external power source via a wire running through or along the handle.

2. The apparatus of claim 1 further comprising:
a mirror attached to the handle at the first end at an angle, the mirror having a mirror base and a mirror surface, the mirror surface facing toward the light source.

3. The apparatus of claim 2 wherein the mirror comprises:
a second light source attached to the mirror base.

4. The apparatus of claim 1 further comprising:
an attachment mechanism to attach the handle to a suction tip.

5. The apparatus of claim 4 wherein the attachment mechanism comprises:
a plurality of clips positioned along the handle.

6. The apparatus of claim 1 further comprising:
a second light source attached to the handle at the second end and connected to the wire; and
a switch positioned on the handle to select one of the first and second light sources for illumination.

7. An apparatus comprising:
a bite block;
a holder embedded in the bite block; and
a light source attached to the holder to illuminate an operation field, the light source being powered by a power source.

8. The apparatus of claim 7 wherein the holder is made of a heat dissipating material.

9. The apparatus of claim 7 wherein the light source is a white light emitting diode (LED).

10. The apparatus of claim 9 wherein the light source has an intensity adjusted by an intensity adjuster.

11. The apparatus of claim 7 wherein the bite block is made of soft plastic.

12. An apparatus comprising:
an absorbent pad; and
an illuminator having a light source attached to the absorbent pad to generate light when powered by a power source.

13. The apparatus of claim 12 further comprising:
a pocket to house at least one of the absorbent pad and the illuminator.

14. The apparatus of claim 12 wherein the light source is a white light emitting diode (LED).

15. An apparatus comprising:
first and second biting pads; and
a pillar between the first and second biting pads having a length adjusted according to distance between upper and lower teeth.

16. The apparatus of claim 15 further comprising:
an illuminator having a light source attached to one of the first and second biting pads by a palatal arm.

17. The apparatus of claim 16 wherein the light source is a white light emitting diode (LED).
